(19) United States
(12) Patent Application Publication
(10) Pub. No.: US 2026/0167919 A1
(43) Pub. Date: Jun. 18, 2026

(54) DETECTION DEVICE

(71) Applicant: Japan Display Inc., Tokyo (JP)

(72) Inventors: Kaoru ITO, Tokyo (JP); Norio MAMBA, Tokyo (JP); Daichi ABE, Tokyo (JP); Yoshihiro SEKIGUCHI, Tokyo (JP)

(21) Appl. No.: 19/416,922

(22) Filed: Dec. 11, 2025

(30) Foreign Application Priority Data
Dec. 12, 2024 (JP) 2024-217766

(51) Int. Cl. C12M 1/36; C12M 1/34

(52) U.S. Cl. CPC C12M 41/48; C12M 41/36

(57) ABSTRACT

According to an aspect, a detection device includes: a sensor panel that has a detection area in which a plurality of optical sensors are arranged in a planar configuration; a light source configured to emit uniform light to an object to be detected provided between the light source and the sensor panel; and a control circuit configured to control the sensor panel and the light source. The control circuit is configured to perform an initial setting process to adjust a light emission amount set value of the light source so that a sensor value acquired from the sensor panel falls within a predetermined target set range.

### CROSS-REFERENCE TO RELATED APPLICATION

This application claims the benefit of priority from Japanese Patent Application No. 2024-217766 filed on Dec. 12, 2024, the entire contents of which are incorporated herein by reference.

### BACKGROUND

### 1. Technical Field

What is disclosed herein relates to a detection device.

### 2. Description of the Related Art

Japanese Patent Application Laid-open Publication No. H06-261737 (JP-A-H06-261737) discloses a biosensor that images, using a solid-state image sensing device, changes over time in state of samples to be cultured that are placed in a culture vessel together with a culture medium necessary for their growth. The samples to be cultured are bacteria, biological tissues such as cells, or the like.

To acquire the changes over time in the samples to be cultured using a detection device such as the biosensor described in JP-A-H06-261737 mentioned above, an appropriate culture medium needs to be selected depending on the type of the samples to be cultured and/or the purpose of the detection. Examples of the culture medium include, but are not limited to, standard agar and sheep blood agar. Since light transmittance differs depending on the type of the culture medium, detection values in a detection plane are required to fall within a detection range of the sensor in order to improve the accuracy of detection of the samples to be cultured.

For the foregoing reasons, there is a need for a detection device capable of increasing the accuracy of detection regardless of the type of the culture medium.

### SUMMARY

According to an aspect, a detection device includes: a sensor panel that has a detection area in which a plurality of optical sensors are arranged in a planar configuration; a light source configured to emit uniform light to an object to be detected provided between the light source and the sensor panel; and a control circuit configured to control the sensor panel and the light source. The control circuit is configured to perform an initial setting process to adjust a light emission amount set value of the light source so that a sensor value acquired from the sensor panel falls within a predetermined target set range.

### BRIEF DESCRIPTION OF THE DRAWINGS

FIG. 1 is a diagram illustrating a main configuration of a detection device according to an embodiment of the present disclosure;

FIG. 2 is a diagram illustrating a configuration example of a detection area and a wiring area;

FIG. 3 is a circuit diagram illustrating a circuit configuration of an optical sensor;

FIG. 4 is a schematic diagram illustrating a configuration example of a light-emitting element;

FIG. 5 is a schematic view illustrating a positional relation between main components of the detection device and an object to be detected, according to the embodiment;

FIG. 6 is a cross-sectional view of the object to be detected in the schematic view illustrated in FIG. 5;

FIG. 7 is a first diagram for explaining an example of a detection operation in the detection device;

FIG. 8 is a second diagram for explaining the example of the detection operation in the detection device;

FIG. 9 is a flowchart illustrating an example of a scan process in the detection device;

FIG. 10A is a schematic diagram schematically illustrating a distribution of sensor values in a detection plane;

FIG. 10B is a schematic diagram schematically illustrating another distribution of the sensor values in the detection plane;

FIG. 10C is a schematic diagram schematically illustrating still another distribution of the sensor values in the detection plane;

FIG. 11 is a flowchart illustrating an example of an initial setting process in the detection device according to the embodiment;

FIG. 12 is a sub-flowchart illustrating an example of a light emission amount adjustment process;

FIG. 13 is a conceptual diagram illustrating a relation between a current value supplied to the light-emitting element and a light emission parameter;

FIG. 14 is a schematic plan view illustrating an example of the optical sensors that acquire the sensor values in the initial setting process;

FIG. 15 is a conceptual diagram for explaining a sensor target value in the light emission amount adjustment process illustrated in FIG. 12;

FIG. 16 is a schematic diagram for explaining an operation in the light emission amount adjustment process illustrated in FIG. 12;

FIG. 17 is a schematic diagram schematically illustrating a configuration example of a detection system provided as a configuration including the detection device; and

FIG. 18 is a schematic diagram illustrating a relation between one detection device and an external configuration in the detection system illustrated in FIG. 17.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

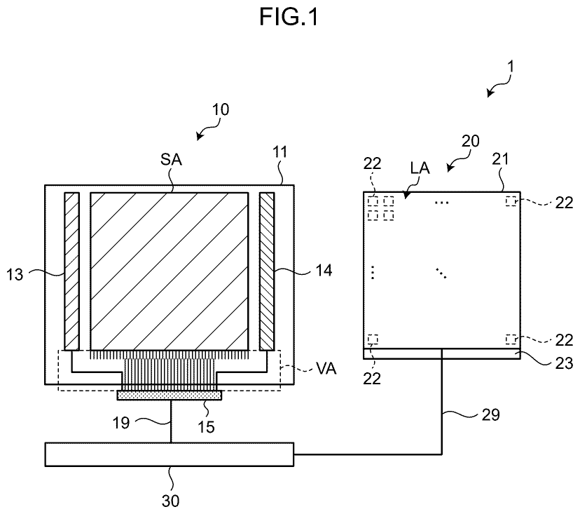

FIG. 1 is a diagram illustrating a main configuration of a detection device 1 according to an embodiment of the present disclosure. The detection device 1 includes a sensor panel 10, a light source panel 20, and a control circuit 30. The sensor panel 10 and the light source panel 20 of the detection device 1 are coupled to the control circuit 30.

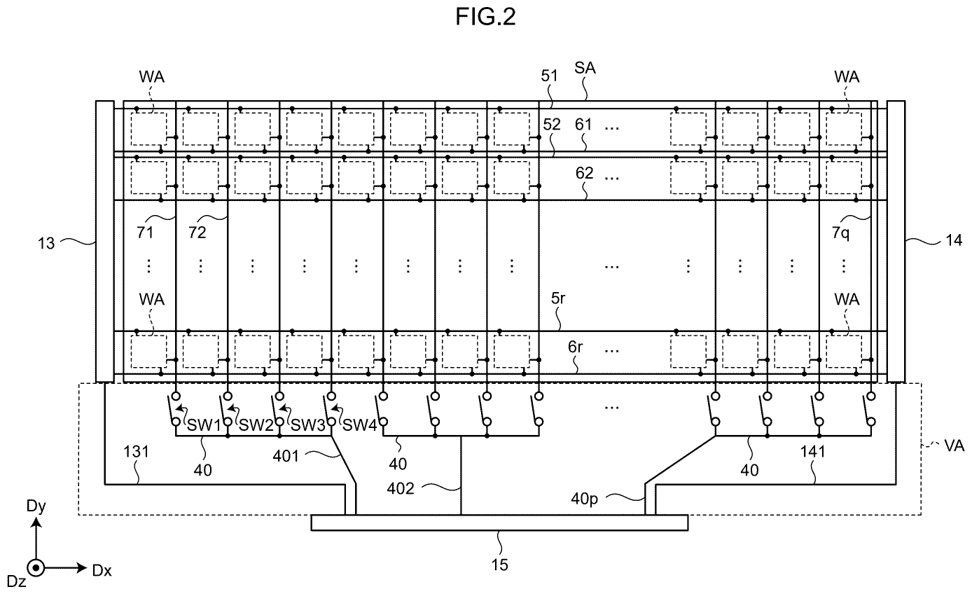

The sensor panel 10 is provided with a detection area SA (refer to FIG. 2) on a substrate 11. A reset circuit 13, a readout circuit 14, and a wiring area VA are provided on the substrate 11. Components on the detection area SA, the reset circuit 13, and the readout circuit 14 are coupled to a detection circuit 15 via the wiring area VA.

The light source panel 20 has a light-emitting area LA that evenly emits light to the detection area SA. As an exemplary aspect, the light source panel 20 is provided with a plurality of light-emitting elements on a substrate, and evenly emits light to the detection area SA using a diffuser plate 21, but the light source panel 20 is not limited to this aspect. A light-emitting element 22 is, for example, a light-emitting diode (LED), and is located in the light-emitting area LA. In the example illustrated in FIG. 1, a plurality of the light-emitting elements 22 are arranged in a matrix having a row-column configuration.

The light source panel 20 is provided with a light source drive circuit 23. Under the control of the control circuit 30, the light source drive circuit 23 controls whether to turn on each of the light-emitting elements 22 and the luminance thereof when being turned on. The light-emitting elements 22 may be provided so as to be individually controllable in light emission, or may be provided so as to emit light collectively.

The control circuit 30 performs various types of control related to the operation of the detection device 1. Specifically, the control circuit 30 is a circuit, such as a field-programmable gate array (FPGA) or an application-specific integrated circuit (ASIC) that can implement a plurality of functions. The control circuit 30 is coupled to the detection circuit 15 via wiring 19 and obtains an output from the detection circuit 15. The control circuit 30 is coupled to the light source drive circuit 23 via wiring 29 and performs processing related to the lighting of the light-emitting elements 22, such as determination of lighting patterns of the light-emitting elements 22.

The control circuit 30 also performs processing related to detection of an object to be detected SUB (refer to FIG. 4) described later. This processing will be described later.

Although not illustrated in the drawings, the detection device 1 includes an analog-to-digital conversion circuit, a digital-to-analog conversion circuit, and other components. The analog-to-digital conversion circuit allows an output from an optical sensor WA (refer to FIG. 2) transmitted through the detection circuit 15 to be handled by arithmetic processing by the control circuit 30. The digital-to-analog conversion circuit makes digital signals generated by the arithmetic processing of the control circuit 30 usable for controlling operations of the sensor panel 10 and the light source panel 20. These circuits may be included, for example, in part or in whole in the control circuit 30, may be functions performed by circuits mounted on flexible printed circuits (FPCs) provided as the wiring 19 and the wiring 29, or may be implemented in other ways in the detection device 1.

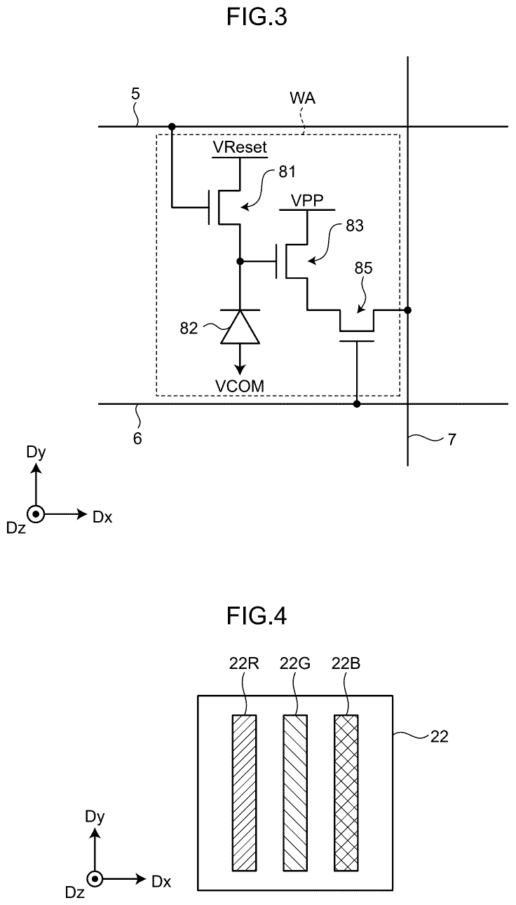

FIG. 2 is a diagram illustrating a configuration example of the detection area SA and the wiring area VA. A plurality of the optical sensors WA (FIG. 3) are provided in the detection area SA. Specifically, the example illustrated in FIG. 2 exemplifies an aspect in which the optical sensors WA are arranged in a matrix having a row-column configuration along a first direction Dx and a second direction Dy. The first direction Dx is orthogonal to the second direction Dy. In the following description, the term “third direction Dz” refers to a direction orthogonal to the first direction Dx and the second direction Dy.

The reset circuit 13 is coupled to reset control lines 51, 52, . . . , 5r. Hereinafter, the term “reset control line 5” refers to any one of the reset control lines 51, 52, . . . , 5r. The reset control line 5 is wiring along the first direction Dx. In the example illustrated in FIG. 2, r reset control lines 5 are arranged in the second direction Dy. r is a natural number equal to or larger than 2. The r reset control lines 5 are coupled, at first ends in the first direction Dx, to the reset circuit 13.

The readout circuit 14 is coupled to readout control lines 61, 62, . . . , 6r. Hereinafter, the term “readout control line 6” refers to any one of the readout control lines 61, 62, . . . , 6r. The readout control line 6 is wiring along the first direction Dx. In the example illustrated in FIG. 2, r readout control lines 6 are arranged in the second direction Dy. The r readout control lines 6 are coupled, at the second ends in the first direction Dx, to the readout circuit 14.

As illustrated in FIG. 2, the reset control lines 5 and the readout control lines 6 are alternately arranged in the second direction Dy in the detection area SA. The reset circuit 13 and the readout circuit 14 illustrated in FIGS. 1 and 2 are arranged at locations facing each other with the detection area SA interposed therebetween, but the layout of the reset circuit 13 and the readout circuit 14 is not limited to this layout and can be changed as appropriate.

Signal lines 71, 72, . . . , 7q are also provided in the detection area SA. Hereinafter, the term “signal line 7” refers to any one of the signal lines 71, 72, . . . , 7q. The signal line 7 is wiring along the second direction Dy.

In the example illustrated in FIG. 2, q signal lines 7 are arranged in the first direction Dx. q is a natural number equal to or larger than 2. The q signal lines 7 are each coupled, at one end in the second direction Dy, to one of a plurality of switches (for example, switch SW1, SW2, SW3, or SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in the one multiplexer 40 is on (conducting state), the other switches are off (non-conducting state). The number of the multiplexers 40 depends on the number (q) of the signal lines 7. When the number of the switches is p, q/p is sufficient as the number of the multiplexers 40. When more than one multiplexers 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the readout circuit 14 is coupled to the detection circuit 15 via wiring 141.

In detecting light using a photodiode 82 (refer to FIG. 3) provided in the optical sensor WA, the detection circuit 15 controls operation timing of the reset circuit 13 and the readout circuit 14. The detection circuit 15 receives the output from the optical sensor WA. The detection circuit 15 converts the signal received from the optical sensor WA into data that can be interpreted by the control circuit 30 and outputs the data to the control circuit 30. Hereafter, a detection value of each of the optical sensors WA output from the detection circuit 15 is also referred to as a “sensor value Raw”. The detection circuit 15 is, for example, a microcontroller unit (MCU) or a readout integrated circuit (ROIC) that includes an analog front-end circuit (AFE).

FIG. 3 is a circuit diagram illustrating a circuit configuration of the optical sensor WA. The first direction Dx and the second direction Dy in FIG. 3 merely correspond to the directions of the reset control line 5, the readout control line 6, and the signal line 7, and do not exactly indicate the relative positional relation of the circuit configuration in the optical sensor WA.

As illustrated in FIG. 3, a reset transistor 81, the photodiode 82, a source follower transistor 83, and a readout transistor 85 are provided in the optical sensor WA. In other words, the reset transistor 81, the source follower transistor 83, and the readout transistor 85 are provided correspondingly to one photodiode 82. The transistors included in the optical sensor WA are each configured as an n-type thin-film transistor (TFT). However, each of the transistors is not limited thereto, and may be configured as a p-type TFT.

A reference potential VCOM is applied to the anode of the photodiode 82. The cathode of the photodiode 82 is coupled to the gate of the source follower transistor 83 and one of the source and the drain of the reset transistor 81.

The gate of the reset transistor 81 is coupled to the reset control line 5. The other of the source and the drain of the reset transistor 81 is supplied with a reset potential VReset. When the reset transistor 81 is turned on (conducting state), the reset potential VReset is supplied to the cathode of the photodiode 82, and the potential of the cathode of the photodiode 82 is reset to the reset potential VReset. The reference potential VCOM is lower than the reset potential VReset. As a result, the photodiode 82 is driven into a reverse bias state.

The source follower transistor 83 is coupled between a terminal supplied with a source-of-output potential VPP and the readout transistor 85. The gate of the source follower transistor 83 is coupled to the cathode of the photodiode 82. The gate of the source follower transistor 83 is supplied with a voltage corresponding to a received light intensity of the photodiode 82. As a result, the source follower transistor 83 outputs a potential corresponding to the received light intensity of the photodiode 82 to the readout transistor 85.

The reset potential VReset, the reference potential VCOM, and the source-of-output potential VPP are supplied by the detection circuit 15 to the optical sensor WA based on, for example, electric power supplied via a power supply circuit (not illustrated) coupled to the detection circuit 15, but are not limited to being supplied in this way, and may be supplied in a different way as appropriate.

The readout transistor 85 is coupled between the source of the source follower transistor 83 and the signal line 7. The gate of the readout transistor 85 is coupled to the readout control line 6. When the readout transistor 85 is turned on (conducting state), the signal output from the source follower transistor 83, that is, the potential corresponding to the received light intensity of the photodiode 82 is output to the signal line 7.

In FIG. 3, the reset transistor 81 and the readout transistor 85 each have a single-gate structure. However, the reset transistor 81 and the readout transistor 85 may each have what is called a double-gate structure configured by coupling two transistors in series, or may have a configuration in which three or more transistors are coupled in series. The circuit of one optical sensor WA is not limited to the configuration including the three transistors of the reset transistor 81, the source follower transistor 83, and the readout transistor 85. The optical sensor WA may have a configuration including two transistors, or four or more transistors.

The reset circuit 13 is a circuit that drives the reset control lines 5 in the detection area SA. The reset circuit 13 includes a shift register circuit, for example.

In the present disclosure, the reset circuit 13 sequentially selects the reset control lines 5 based on various control signals such as start pulse signals and clock pulse signals supplied from the detection circuit 15, and supplies a reset control signal RST to the selected reset control lines 5. In other words, the reset circuit 13 simultaneously supplies the reset control signal RST to the optical sensors WA arranged in the first direction Dx, and sequentially supplies the reset control signal RST to the optical sensors WA arranged in the second direction Dy. This operation resets the potentials of the photodiodes 82 of the optical sensors WA coupled to the reset control lines 5 selected by the reset circuit 13 for the optical sensors WA.

The readout circuit 14 is a circuit that drives the readout control lines 6 in the detection area SA. The readout circuit 14 includes a shift register circuit, for example.

In the present disclosure, the readout circuit 14 sequentially selects the readout control lines 6 based on the various control signals such as the start pulse signals and the clock pulse signals supplied from the detection circuit 15, and supplies a readout control signal RD to the selected readout control lines 6. In other words, the readout circuit 14 simultaneously supplies the readout control signal RD to the optical sensors WA arranged in the first direction Dx, and sequentially supplies the readout control signal RD to the optical sensors WA arranged in the second direction Dy. As a result, the potentials of the optical sensors WA coupled to the readout control lines 6 selected by the readout circuit 14 are read out.

FIG. 4 is a schematic diagram illustrating a configuration example of the light-emitting element 22. As illustrated in FIG. 4, the light-emitting element 22 includes a first light-emitting element 22R, a second light-emitting element 22G, and a third light-emitting element 22B. The first light-emitting element 22R, the second light-emitting element 22G, and the third light-emitting element 22B emit light in different colors from one another. Specifically, the first light-emitting element 22R emits red (R) light, the second light-emitting element 22G emits green (G) light, and the third light-emitting element 22B emits blue (B) light. In this case, white light is emitted by simultaneously turning on the first light-emitting element 22R, the second light-emitting element 22G, and the third light-emitting element 22B.

FIG. 4 illustrates an exemplary configuration in which the longitudinal directions of the first light-emitting element 22R, the second light-emitting element 22G, and the third light-emitting element 22B extend along the second direction Dy, and the first light-emitting element 22R, the second light-emitting element 22G, and the third light-emitting element 22B are arranged in this order from one side to the other side in the first direction Dx. However, the shapes and positional relation of the first light-emitting element 22R, the second light-emitting element 22G, and the third light-emitting element 22B from a planar viewpoint are not limited to this exemplary configuration, and can be changed as appropriate. A single light-emitting element that emits white (W) light may be provided instead of the first light-emitting element 22R, the second light-emitting element 22G, and the third light-emitting element 22B.

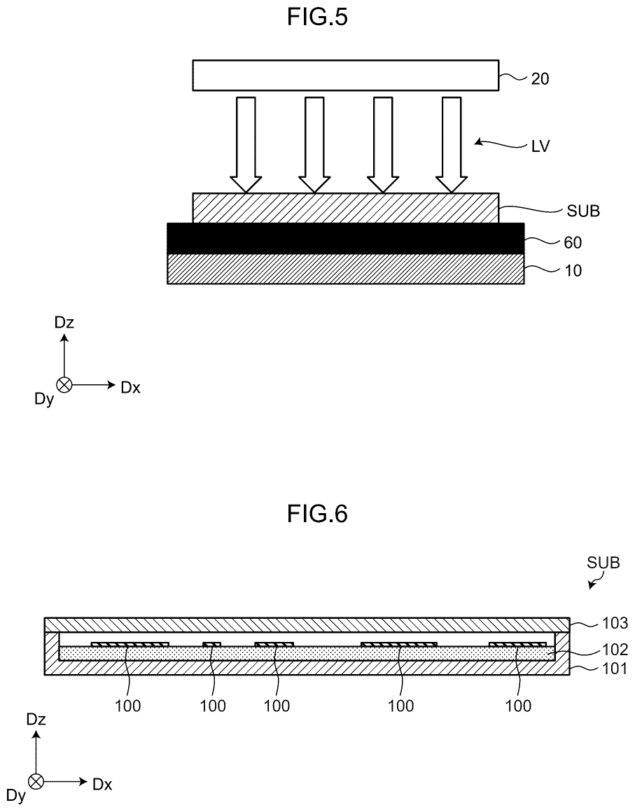

FIG. 5 is a schematic view illustrating a positional relation between main components of the detection device 1 and an object to be detected SUB, according to the embodiment. FIG. 6 is a cross-sectional view of the object to be detected SUB in the schematic view illustrated in FIG. 5. In the detection device 1, the light source panel 20 and the sensor panel 10 are provided so as to face each other in the third direction Dz with the object to be detected SUB interposed therebetween.

As illustrated in FIG. 6, the object to be detected SUB is provided with a cover member 103 that is placed on the upper side of a light-transmitting placement substrate 101 formed of glass, for example, to cover a plurality of samples to be cultured 100. More specifically, the placement substrate 101 and the cover member 103 are a Petri dish, for example. The samples to be cultured 100 are placed on the placement substrate 101 together with a culture medium 102 (e.g., agar) and placed between the sensor panel 10 and the light source panel 20.

In the present disclosure, the samples to be cultured 100 are, for example, bacteria or biological tissues such as cells. The culture medium 102 for culturing the samples to be cultured 100 is exemplified by, for example, standard agar or sheep blood agar. Different types of the culture medium 102 have different light transmittance. Specifically, the sheep blood agar has relatively lower light transmittance than the standard agar.

A light directivity control element 60 is provided between the object to be detected SUB and the sensor panel 10. The light directivity control element 60 is an optical element that transmits, toward the photodiode 82, components of the light emitted from the light source panel 20 that travel in a direction orthogonal to the sensor panel 10. The light directivity control element 60 is also called collimating apertures or a collimator. Alternatively, the light directivity control element 60 may be configured with a louver or microlenses instead of the collimator.

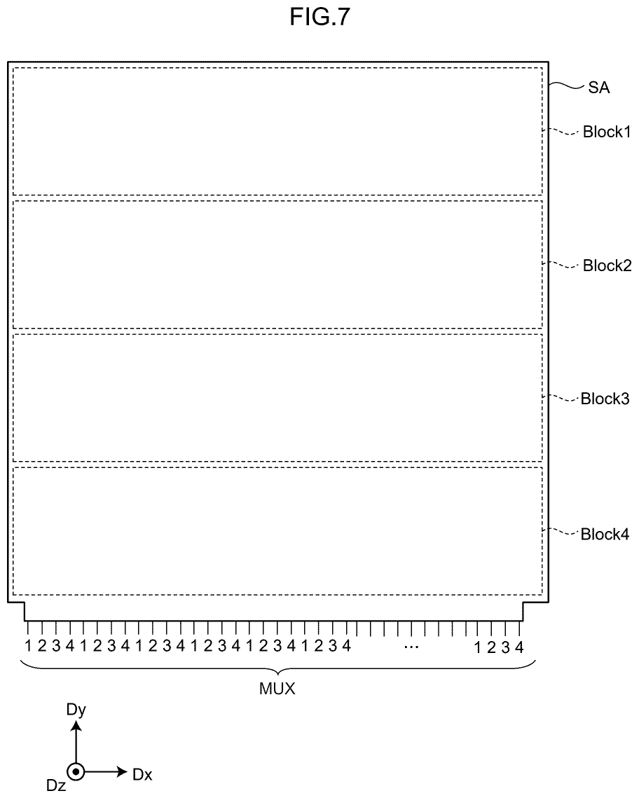

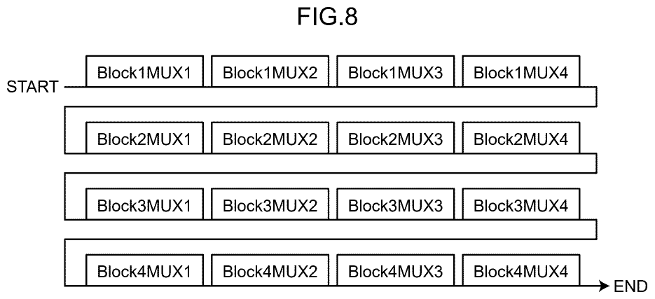

The following describes a specific example of a detection operation in the detection device 1. FIG. 7 is a first diagram for explaining the example of the detection operation in the detection device 1. FIG. 8 is a second diagram for explaining the example of the detection operation in the detection device 1.

In an aspect illustrated in FIGS. 7 and 8, the detection area SA is divided into a plurality of segmented areas (blocks) in the second direction Dy. The example illustrated in FIG. 7 exemplifies the aspect in which the detection area SA is divided into four segmented areas Block1, Block2, Block3, and Block4. The number of the optical sensors WA arranged in the second direction Dy is preferably the same in each of the segmented areas Block1, Block2, Block3, and Block4.

In the aspect illustrated in FIGS. 7 and 8, MUX1, MUX2, MUX3, and MUX4 correspond to the switches SW1, SW2, SW3, and SW4 (refer to FIG. 2) included in each of the multiplexers 40.

In the detection device 1 according to such an aspect, the sensor values Raw are acquired in the following order:
“Block1MUX1”, “Block1MUX2”, “Block1MUX3”, “Block1MUX4”, “Block2MUX1”, “Block2MUX2”, “Block2MUX3”, “Block2MUX4”, “Block3MUX1”, “Block3MUX2”, “Block3MUX3”, “Block3MUX4”, “Block4MUX1”, “Block4MUX2”, “Block4MUX3”, and “Block4MUX4”, as illustrated in FIG. 8.

In “Block1MUX1” illustrated in FIG. 8, the sensor values Raw of the optical sensors WA coupled to the detection circuit 15 via the switch SW1 of each of the multiplexers 40 in the segmented area Block1 are sequentially acquired while the switch SW1 of the multiplexer 40 is controlled to be on (conducting state).

In “Block1MUX2” illustrated in FIG. 8, the sensor values Raw of the optical sensors WA coupled to the detection circuit 15 via the switch SW2 of each of the multiplexers 40 in the segmented area Block1 are sequentially acquired while the switch SW2 of the multiplexer 40 is controlled to be on (conducting state).

In “Block1MUX3” illustrated in FIG. 8, the sensor values Raw of the optical sensors WA coupled to the detection circuit 15 via the switch SW3 of each of the multiplexers 40 in the segmented area Block1 are sequentially acquired while the switch SW3 of the multiplexer 40 is controlled to be on (conducting state).

In “Block1MUX4” illustrated in FIG. 8, the sensor values Raw of the optical sensors WA coupled to the detection circuit 15 via the switch SW4 of each of the multiplexers 40 in the segmented area Block1 are sequentially acquired while the switch SW4 of the multiplexer 40 is controlled to be on (conducting state).

In “Block2MUX1” illustrated in FIG. 8, the sensor values Raw of the optical sensors WA coupled to the detection circuit 15 via the switch SW1 of each of the multiplexers 40 in the segmented area Block2 are sequentially acquired while the switch SW1 of the multiplexer 40 is controlled to be on (conducting state).

In “Block2MUX2” illustrated in FIG. 8, the sensor values Raw of the optical sensors WA coupled to the detection circuit 15 via the switch SW2 of each of the multiplexers 40 in the segmented area Block2 are sequentially acquired while the switch SW2 of the multiplexer 40 is controlled to be on (conducting state).

In “Block2MUX3” illustrated in FIG. 8, the sensor values Raw of the optical sensors WA coupled to the detection circuit 15 via the switch SW3 of each of the multiplexers 40 in the segmented area Block2 are sequentially acquired while the switch SW3 of the multiplexer 40 is controlled to be on (conducting state).

In “Block2MUX4” illustrated in FIG. 8, the sensor values Raw of the optical sensors WA coupled to the detection circuit 15 via the switch SW4 of each of the multiplexers 40 in the segmented area Block2 are sequentially acquired while the switch SW4 of the multiplexer 40 is controlled to be on (conducting state).

In “Block3MUX1” illustrated in FIG. 8, the sensor values Raw of the optical sensors WA coupled to the detection circuit 15 via the switch SW1 of each of the multiplexers 40 in the segmented area Block3 are sequentially acquired while the switch SW1 of the multiplexer 40 is controlled to be on (conducting state).

In “Block3MUX2” illustrated in FIG. 8, the sensor values Raw of the optical sensors WA coupled to the detection circuit 15 via the switch SW2 of each of the multiplexers 40 in the segmented area Block3 are sequentially acquired while the switch SW2 of the multiplexer 40 is controlled to be on (conducting state).

In “Block3MUX3” illustrated in FIG. 8, the sensor values Raw of the optical sensors WA coupled to the detection circuit 15 via the switch SW3 of each of the multiplexers 40 in the segmented area Block3 are sequentially acquired while the switch SW3 of the multiplexer 40 is controlled to be on (conducting state).

In “Block3MUX4” illustrated in FIG. 8, the sensor values Raw of the optical sensors WA coupled to the detection circuit 15 via the switch SW4 of each of the multiplexers 40 in the segmented area Block3 are sequentially acquired while the switch SW4 of the multiplexer 40 is controlled to be on (conducting state).

In “Block4MUX1” illustrated in FIG. 8, the sensor values Raw of the optical sensors WA coupled to the detection circuit 15 via the switch SW1 of each of the multiplexers 40 in the segmented area Block4 are sequentially acquired while the switch SW1 of the multiplexer 40 is controlled to be on (conducting state).

In “Block4MUX2” illustrated in FIG. 8, the sensor values Raw of the optical sensors WA coupled to the detection circuit 15 via the switch SW2 of each of the multiplexers 40 in the segmented area Block4 are sequentially acquired while the switch SW2 of the multiplexer 40 is controlled to be on (conducting state).

In “Block4MUX3” illustrated in FIG. 8, the sensor values Raw of the optical sensors WA coupled to the detection circuit 15 via the switch SW3 of each of the multiplexers 40 in the segmented area Block4 are sequentially acquired while the switch SW3 of the multiplexer 40 is controlled to be on (conducting state).

In “Block4MUX4” illustrated in FIG. 8, the sensor values Raw of the optical sensors WA coupled to the detection circuit 15 via the switch SW4 of each of the multiplexers 40 in the segmented area Block4 are sequentially acquired while the switch SW4 of the multiplexer 40 is controlled to be on (conducting state).

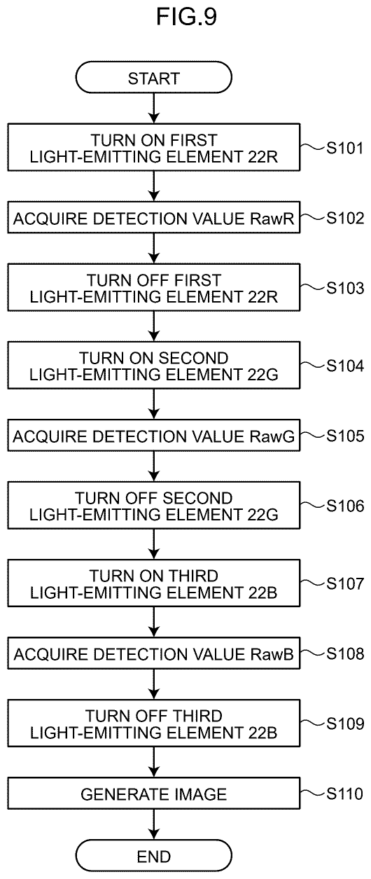

FIG. 9 is a flowchart illustrating an example of a scan process in the detection device 1. In the present disclosure, the term “scan process” refers to a process to generate an image of the object to be detected SUB by turning on the light source panel 20 to emit the light to the sensor panel 10 and acquiring the sensor value of each of the optical sensors WA corresponding to the amount of light received by the photodiode 82 included in the optical sensor WA.

In the scan process with reference to FIG. 9, the control circuit 30 first turns on the first light-emitting elements 22R (Step S101), acquires a sensor value RawR of each of the optical sensors WA (Step S102), and turns off the first light-emitting elements 22R (Step S103).

The control circuit 30 then turns on the second light-emitting elements 22G (Step S104), acquires a sensor value RawG of each of the optical sensors WA (Step S105), and turns off the second light-emitting elements 22G (Step S106).

The control circuit 30 then turns on the third light-emitting elements 22B (Step S107), acquires a sensor value RawB for each of the optical sensors WA (Step S108), and turns off the third light-emitting elements 22B (Step S109).

Then, the control circuit 30 generates the image of the object to be detected SUB in the plane of the detection area SA by combining the acquired sensor values RawR, RawG, and RawB of the respective optical sensors WA (Step S110).

By performing the scan process described above at intervals of a predetermined wait time, changes over time of the state of the samples to be cultured 100 can be acquired. The wait time in the present disclosure is five minutes, for example.

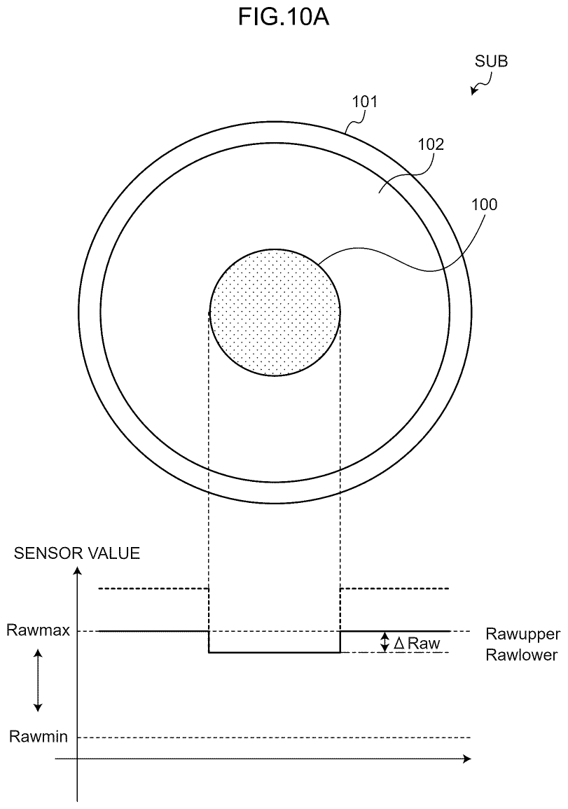

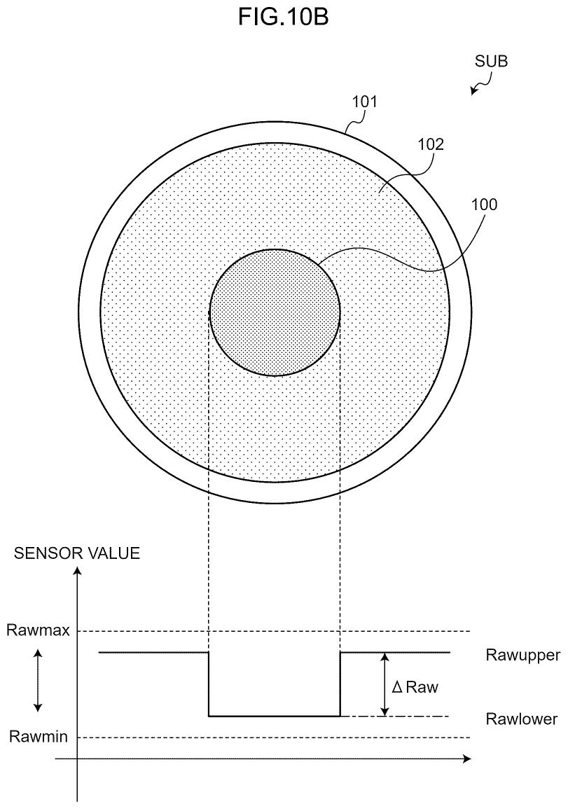

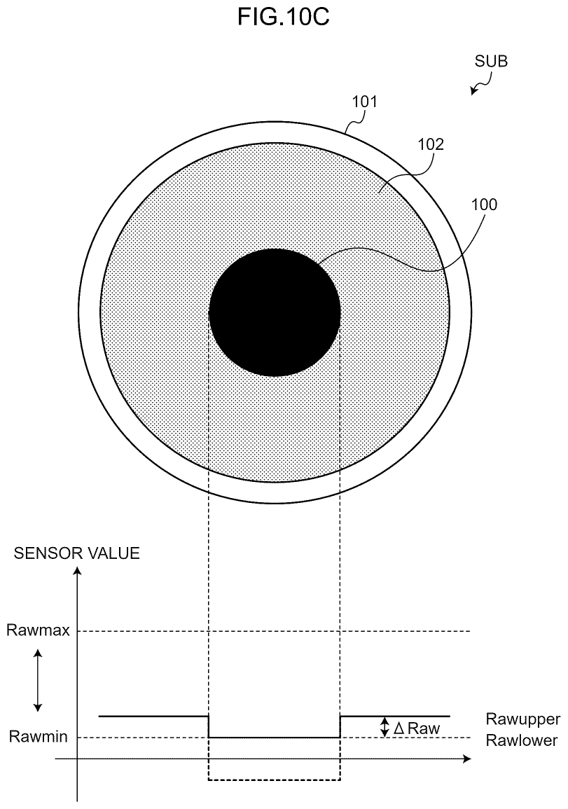

FIGS. 10A, 10B, and 10C are schematic diagrams schematically illustrating distributions of the sensor values Raw in a detection plane. FIGS. 10A, 10B, and 10C each illustrate an aspect in which one sample to be cultured 100 is present in the center of the culture medium 102. The light transmittance of the culture medium 102 decreases in the order of FIGS. 10A, 10B, and 10C.

Rawmin illustrated in FIGS. 10A, 10B, and 10C represents the lower limit of the sensor value (hereinafter, also referred to as a “lower limit sensor value Rawmin”) in the detection circuit 15. Rawmax illustrated in FIGS. 10A, 10B, and 10C represents the upper limit of the sensor value (hereinafter, also referred to as an “upper limit sensor value Rawmax”) in the detection circuit 15. In the present disclosure, a range from the lower limit sensor value Rawmin to the upper limit sensor value Rawmax is defined as being within a detection range in the detection circuit 15.

The example illustrated in FIG. 10B illustrates an aspect in which a sensor value Rawupper in an area corresponding to the culture medium 102 and a sensor value Rawlower in an area corresponding to the sample to be cultured 100 fall within the detection range in the detection circuit 15. More specifically, in the example illustrated in FIG. 10B, the sensor value Rawlower in the area corresponding to the sample to be cultured 100 is equal to or higher than the lower limit sensor value Rawmin (Rawlower≥Rawmin), and the sensor value Rawupper in the area corresponding to the culture medium 102 is equal to or lower than the upper limit sensor value Rawmax (Rawupper≤Rawmax).

In contrast, if the light transmittance of the culture medium 102 is relatively higher, the sensor value Rawupper in the area corresponding to the culture medium 102 is limited to the upper limit sensor value Rawmax, as illustrated in FIG. 10A. As a result, a difference ΔRaw relative to the sensor value Rawlower in the area corresponding to the sample to be cultured 100 becomes smaller.

If the light transmittance of the culture medium 102 is relatively lower, the sensor value Rawlower in the area corresponding to the sample to be cultured 100 is limited to the lower limit sensor value Rawmin, as illustrated in FIG. 10C. As a result, the difference ΔRaw relative to the sensor value Rawupper in the area corresponding to the culture medium 102 becomes smaller.

Thus, depending on the type of the culture medium 102, changes over time in state of the sample to be cultured may not be normally acquired.

In the present embodiment, the amount of light LV (refer to FIG. 5) emitted from the light-emitting area LA of the light source panel 20 to the detection area SA of the sensor panel 10 is adjusted in an initial setting process before the scan process starts, and, when the subsequent scan process is performed, each of the light-emitting elements 22 is driven at the light emission amount set in the initial setting process. The following describes a specific example of the initial setting process in the detection device 1 according to the embodiment.

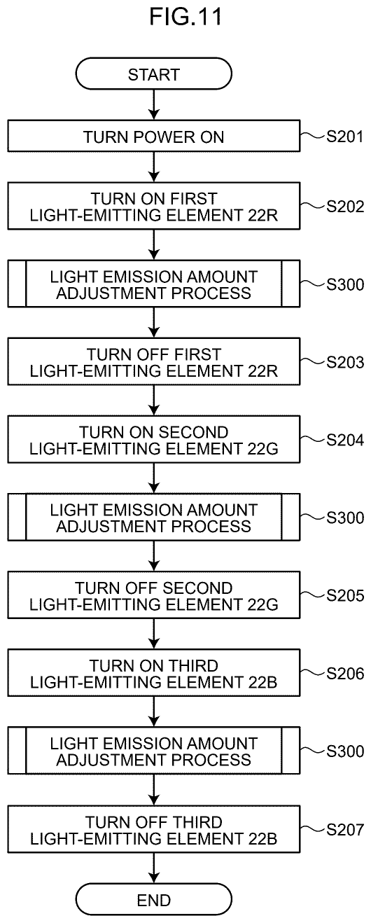

FIG. 11 is a flowchart illustrating an example of the initial setting process in the detection device 1 according to the embodiment. The initial setting process illustrated in FIG. 11 is performed while the samples to be cultured 100 are not present on the culture medium 102 before the start of the scan process illustrated in FIG. 9, as described above.

Specifically, when the power of the detection device 1 is turned on (Step S201), the control circuit 30 first turns on the first light-emitting elements 22R (Step S202), and performs a light emission amount adjustment process for the first light-emitting elements 22R (Step S300).

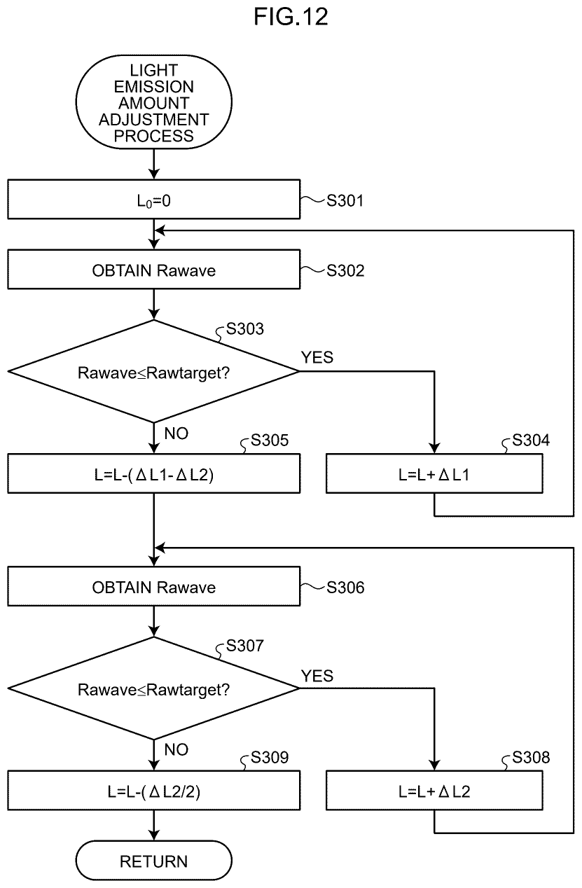

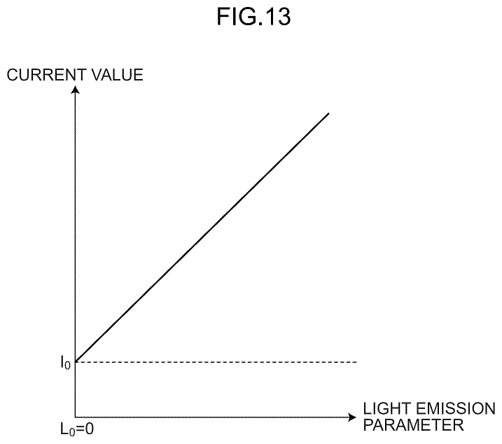

FIG. 12 is a sub-flowchart illustrating an example of the light emission amount adjustment process. In the light emission amount adjustment process illustrated in FIG. 12, the control circuit 30 first initializes the current value supplied to the light-emitting elements 22 (in this case, the first light-emitting elements 22R). More specifically, the control circuit 30 sets a light emission parameter L corresponding to the current value supplied to the light-emitting elements 22 to an initial value L₀ (L=L₀, Step S301). FIG. 13 is a conceptual diagram illustrating a relation between the current value supplied to the light-emitting element 22 and the light emission parameter.

In the present disclosure, the light emission parameter L is a discrete value indicated by, for example, an 8-bit digital value. The initial value L₀ of the light emission parameter L is set to “0” for example. An initial current amount I₀ corresponding to the initial value L₀ of the light emission parameter L and a current difference value per step of the light emission parameter L have been preset.

The control circuit 30 reads out the current amount I₀ corresponding to the initial value L₀ of the light emission parameter L, and supplies the read out current amount I₀ to the light-emitting elements 22 (in this case, the first light-emitting elements 22R) of the light source panel 20.

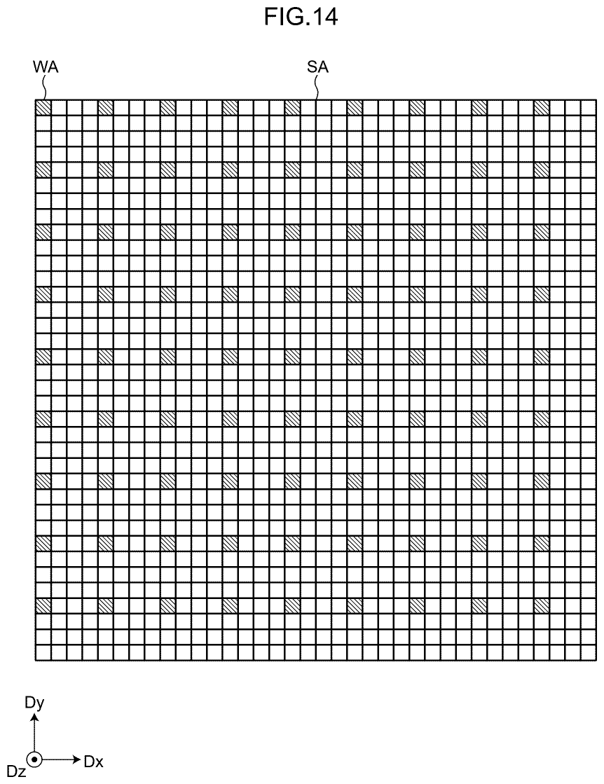

The control circuit 30 then obtains an average value Rawave (hereinafter, also referred to as a “sensor value Rawave”) of the sensor values Raw of the respective optical sensors WA in the detection area SA (Step S302). The sensor value Rawave obtained in the initial setting process may be, for example, the average value of the sensor values Raw of some of the optical sensors WA in the detection area SA, as illustrated in FIG. 14.

FIG. 14 is a schematic plan view illustrating an example of the optical sensors WA that acquire the sensor values Raw in the initial setting process. In the example illustrated in FIG. 14, the optical sensors WA that acquire the sensor values Raw are indicated by hatching.

FIG. 14 illustrates an aspect in which the sensor values Raw of the optical sensors WA apart from one another by predetermined distances in the first direction Dx and the second direction Dy in the detection area SA are acquired by intermittently driving the optical sensors WA arranged in the first direction Dx and the optical sensors WA arranged in the second direction Dy, and thus, the number of the optical sensors WA that acquire the sensor values Raw is reduced to 1/16 the total number of the optical sensors WA.

An alternative aspect may be employed such that, for example, the sensor values Raw of the respective optical sensors WA in the segmented areas Block2 and Block3 illustrated in FIG. 7 are acquired, and the average value of the respective sensor values Raw in the segmented areas Block2 and Block3 is calculated as the sensor value Rawave. Thus, by reducing the number of the optical sensors WA that acquire the sensor values Raw in the light emission amount adjustment process illustrated in FIG. 12, the time required for the light emission amount adjustment process for the light source panel 20 illustrated in FIG. 11 can be reduced.

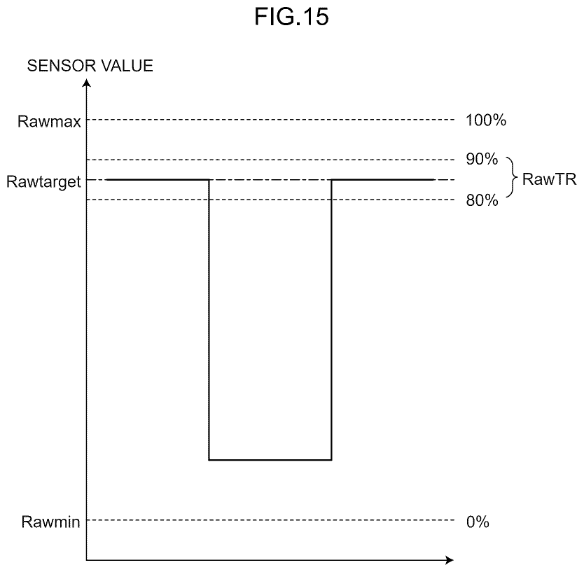

Referring back to FIG. 12, the control circuit 30 determines whether the sensor value Rawave is equal to or lower than a sensor target value Rawtarget (Step S303). FIG. 15 is a conceptual diagram for explaining the sensor target value Rawtarget in the light emission amount adjustment process illustrated in FIG. 12.

The sensor target value Rawtarget in the light emission amount adjustment process illustrated in FIG. 12 has been preset within a target set range RawTR ranging from 80% to 90% of the upper limit sensor value Rawmax in the detection circuit 15, as illustrated in FIG. 15. As described above, the initial setting process illustrated in FIG. 11 is performed while the samples to be cultured 100 are almost non-existent on the culture medium 102 before the start of the scan process illustrated in FIG. 9. Therefore, the sensor value Rawave can roughly be considered to be the sensor value Rawupper in the area corresponding to the culture medium 102. By setting the sensor target value Rawtarget within the target set range RawTR ranging from 80% to 90% of the upper limit sensor value Rawmax in the detection circuit 15, a margin can be secured for the sensor value Rawlower in the area corresponding to the sample to be cultured 100 when acquiring the changes over time of the state of the sample to be cultured 100.

If the sensor value Rawave is equal to or lower than the sensor target value Rawtarget (Rawave≤Rawtarget; Yes at Step S303), the control circuit 30 adds ΔL1 steps (ΔL1 steps are, for example, 20 steps) to the light emission parameter L (Step S304), and performs again the process starting at Step S302 (first process).

If the sensor value Rawave exceeds the sensor target value Rawtarget (Rawave>Rawtarget; No at Step S303), the control circuit 30 subtracts (ΔL1−ΔL2) steps (ΔL2 steps are, for example, 2 steps, and in this case, (ΔL1−ΔL2) steps are 18 steps) from the light emission parameter L (Step S305), and calculates the sensor value Rawave in the same way as in Step S302 (Step S306) (second process).

Then, in the same way as in Step S303, the control circuit 30 determines whether the sensor value Rawave is equal to or lower than the sensor target value Rawtarget (Step S307). If the sensor value Rawave is equal to or lower than the sensor target value Rawtarget (Rawave≤Rawtarget; Yes at Step S307), the control circuit 30 adds ΔL2 steps to the light emission parameter L (Step S308), and performs again the process starting at Step S306 (third process).

If the sensor value Rawave exceeds the sensor target value Rawtarget (Rawave>Rawtarget; No at Step S307), the control circuit 30 subtracts (ΔL2/2) steps (for example, 1 step) from the light emission parameter L (Step S309), and sets the light emission parameter L at that time point as the light emission parameter (light emission amount set value) that defines a current value to be supplied to each first light-emitting element 22R in the scan process illustrated in FIG. 9 (fourth process).

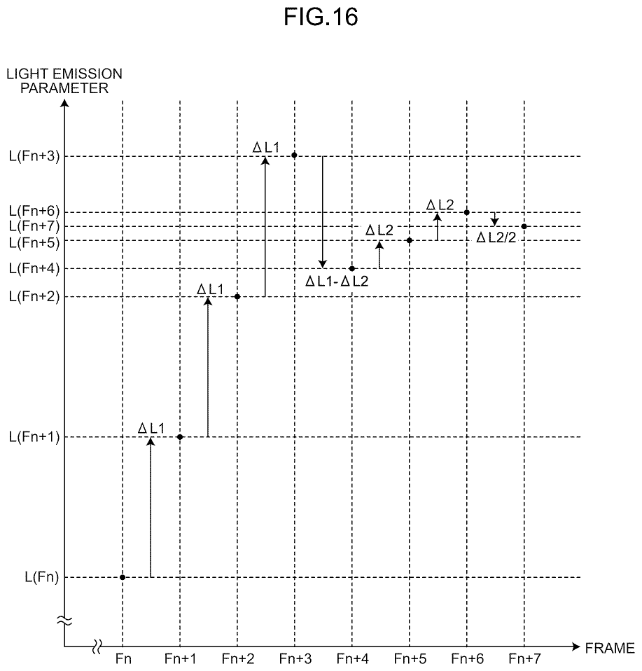

The following describes a specific example of the operation in the light emission amount adjustment process illustrated in FIG. 12. FIG. 16 is a schematic diagram for explaining the operation in the light emission amount adjustment process illustrated in FIG. 12.

As described above, the control circuit 30 changes the light emission parameter L by an increment of a large change step ΔL1 at Step S304 (first process) until the sensor value Rawave exceeds the target value Rawtarget at Step S303 (up to Fn+3 frames in the example illustrated in FIG. 16).

If the sensor value Rawave exceeds the target value Rawtarget (Rawave>Rawtarget; No at Step S303), the control circuit 30 subtracts (ΔL1−ΔL2) steps from the light emission parameter L (Step S305) (second process), and changes the light emission parameter L by an increment of a small change step ΔL2 at Step S308 (third process) until the sensor value Rawave exceeds the target value Rawtarget at Step S307 thereafter (up to Fn+6 frames in the example illustrated in FIG. 16).

If the sensor value Rawave exceeds the target value Rawtarget (Rawave>Rawtarget; No at Step S307), the control circuit 30 subtracts (ΔL2/2) steps from the light emission parameter L (Step S309) (fourth process), and returns to the initial setting process illustrated in FIG. 11.

By appropriately setting the change step ΔL1 of the light emission parameter L for Step S304 and the change step ΔL2 of the light emission parameter L for Step S308 of the light emission amount adjustment process illustrated in FIG. 12, time required for the light emission amount adjustment process illustrated in FIG. 12 can be reduced, and the accuracy of adjustment in the light emission amount adjustment process can be increased.

Referring back to the initial setting process illustrated in FIG. 11, the control circuit 30 turns off the first light-emitting elements 22R (Step S203), then turns on the second light-emitting elements 22G (Step S204), and performs the light emission amount adjustment process for the second light-emitting elements 22G (Step S300). The light emission amount adjustment process for the second light-emitting elements 22G is the same as that for the first light-emitting elements 22R described above, and therefore, will not be described in detail. The control circuit 30 sets the light emission parameter L after the light emission amount adjustment process for the second light-emitting elements 22G as the light emission parameter (light emission amount set value) that defines a current value to be supplied to each second light-emitting element 22G in the scan process illustrated in FIG. 9.

Referring back to the initial setting process illustrated in FIG. 11, the control circuit 30 turns off the second light-emitting elements 22G (Step S205), then turns on the third light-emitting elements 22B (Step S206), and performs the light emission amount adjustment process for the third light-emitting elements 22B (Step S300). The light emission amount adjustment process for the third light-emitting elements 22B is the same as that for the first light-emitting elements 22R and the second light-emitting elements 22G described above, and therefore, will not be described in detail. The control circuit 30 sets the light emission parameter L after the light emission amount adjustment process for the third light-emitting elements 22B as the light emission parameter (light emission amount set value) that defines a current value to be supplied to each third light-emitting element 22B in the scan process illustrated in FIG. 9.

Referring back to the initial setting process illustrated in FIG. 11, the control circuit 30 turns off the third light-emitting elements 22B (Step S207), and ends the initial setting process illustrated in FIG. 11.

When performing the scan process illustrated in FIG. 9, the detection device 1 according to the embodiment drives the respective light-emitting elements 22 of the light source panel 20 by applying thereto the current values corresponding to the light emission parameters (light emission amount set values) that have been set after the initial setting process illustrated in FIG. 11. This operation allows the changes over time of the state of the samples to be cultured 100 to fall within the detection range in the detection circuit 15 regardless of the type of the culture medium 102 (light transmittance).

As described above, in the initial setting process before the scan process starts, the detection device 1 according to the embodiment adjusts the amount of the light LV (refer to FIG. 5) emitted from the light-emitting area LA of the light source panel 20 to the detection area SA of the sensor panel 10. When performing the subsequent scan process, the detection device 1 drives each of the light-emitting elements 22 at the light emission amount set in the initial setting process. This operation allows normal acquisition of the changes over time of the state of the samples to be cultured 100 regardless of the type of the culture medium (light transmittance).

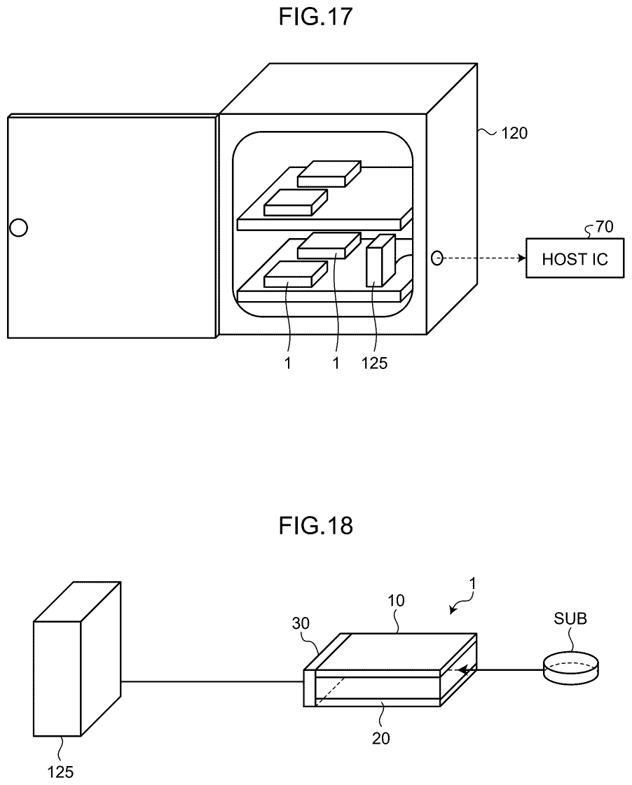

FIG. 17 is a schematic diagram schematically illustrating a configuration example of a detection system including the detection device 1. In the detection system illustrated in FIG. 17, a plurality of the detection devices 1 are electrically coupled to a common host integrated circuit (IC) 70 via a coupling circuit 125.

An incubator 120 is maintained such that an environment (temperature, humidity, and the like) therein is suitable for culturing the object to be detected while a door is closed. In the detection system illustrated in FIG. 17, the detection devices 1 are placed in the incubator 120.

FIG. 18 is a schematic diagram illustrating a relation between one detection device 1 and an external configuration in the detection system illustrated in FIG. 17. As illustrated in FIG. 18, the detection device 1 is coupled to the coupling circuit 125 by coupling the control circuit 30 to the coupling circuit 125. As illustrated in FIG. 18, the sensor panel 10 faces the light source panel 20. A gap where the object to be detected SUB can be placed is provided between the sensor panel 10 and the light source panel 20.

While the preferred embodiment has been described above, the present invention is not limited to such an embodiment. The content disclosed in the embodiment is merely an example, and can be variously modified within the scope not departing from the gist of the present invention. Any modifications appropriately made within the scope not departing from the gist of the present invention also naturally belong to the technical scope of the present invention. At least one of various omissions, substitutions, and changes of the components can be made without departing from the gist of the embodiment described above and the modifications thereof.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a sensor panel that has a detection area in which a plurality of optical sensors are arranged in a planar configuration;
a light source configured to emit uniform light to an object to be detected provided between the light source and the sensor panel; and
a control circuit configured to control the sensor panel and the light source, wherein
the control circuit is configured to perform an initial setting process to adjust a light emission amount set value of the light source so that a sensor value acquired from the sensor panel falls within a predetermined target set range.

2. The detection device according to claim 1, wherein the target set range is a range from 80% to 90% of a detectable upper limit value of the sensor value.

3. The detection device according to claim 1, wherein the control circuit is configured to adjust the light emission amount set value so that an average of a plurality of the sensor values corresponding to a plurality of the optical sensors falls within the predetermined target set range in the initial setting process.

4. The detection device according to claim 1, wherein the control circuit is configured to adjust the light emission amount set value so that an average of a plurality of the sensor values corresponding to some of the optical sensors included in the detection area falls within the predetermined target set range in the initial setting process.

5. The detection device according to claim 1, wherein
a plurality of the optical sensors are arranged in a matrix having a row-column configuration along a first direction and a second direction intersecting the first direction in the detection area of the sensor panel, and
the control circuit is configured to adjust the light emission amount set value so that an average of a plurality of the sensor values corresponding to a plurality of the optical sensors included in the detection area that are apart from one another by predetermined distances in the first direction and the second direction falls within the predetermined target set range in the initial setting process.

6. The detection device according to claim 1, wherein
the detection area of the sensor panel is divided into a plurality of segmented areas, and
the control circuit is configured to adjust the light emission amount set value so that an average of a plurality of the sensor values corresponding to a plurality of the optical sensors included in some of the segmented areas falls within the predetermined target set range in the initial setting process.

7. The detection device according to claim 1, wherein
the light source is configured to emit light in a plurality of colors different from one another in a time-division manner, and
the control circuit is configured to adjust the light emission amount set value for each emission color of the light source in the initial setting process.

8. The detection device according to claim 1, wherein
the initial setting process comprises:
a first process to increase an amount of light of the light source by an increment of a first change step until the sensor value acquired from the sensor panel exceeds a predetermined sensor target value;
a second process to reduce the amount of light of the light source by an amount obtained by subtracting a second change step smaller than the first change step from the first change step when the sensor value acquired from the sensor panel has exceeded the predetermined sensor target value in the first process;
a third process to increase the amount of light of the light source by an increment of the second change step until the sensor value acquired from the sensor panel exceeds the predetermined sensor target value after the second process; and
a fourth process to reduce the amount of light of the light source by half the second change step when the sensor value acquired from the sensor panel has exceeded the predetermined sensor target value in the third process.
